(19) United States
(12) Patent Application Publication
(10) Pub. No.: US 2007/0149780 A1
(43) Pub. Date: Jun. 28, 2007

(54) Process for preparing haloalkyl(thio)vinimidinium salts and 4-(haloalkyl(thio))pyrazoles and their conversion to crop protection compositions

(75) Inventors: Sergiy Pazenok, Solingen (DE); Mark James Ford, Schmitten-Oberreifenberg (DE)

Correspondence Address: CONNOLLY BOVE LODGE & HUTZ, LLP, P O BOX 2207, WILMINGTON, DE, 19899, US

(73) Assignee: Bayer CropScience GmbH, Frankfurt am Main (DE)

(21) Appl. No.: 11/634,691

(22) Filed: Dec. 5, 2006

(30) Foreign Application Priority Data
Dec. 7, 2005 (DE) 10 2005 058 418.7

(51) Int. Cl. C07D 403/02; C07D 231/12; C07C 251/10

(52) U.S. Cl. 544/314; 546/275.4; 548/376.1; 564/279

(57) ABSTRACT

A process for preparing compounds of the formula (IX) by
a) reaction of haloalkyl(thio)acetic acids of the formula (II) with formamides of the formula (III) to give vinimidinium salts of the formula (I), 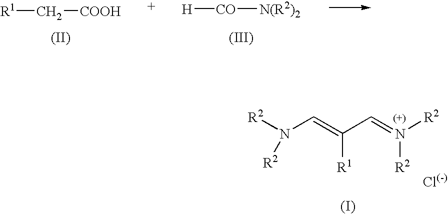
b) reaction of vinimidinium salts of the formula (I) with hydrazines of the formula (V) to give pyrazoles of the formula (IV) 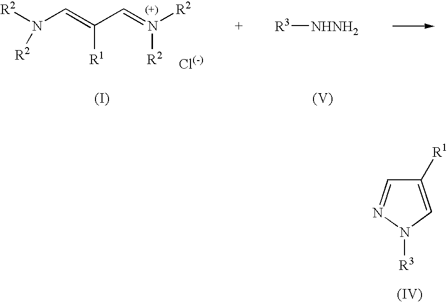
c) base-catalyzed reaction of compounds of the formula (VI) with pyrazoles of the formula (IV) to give compounds of the formula (VII) 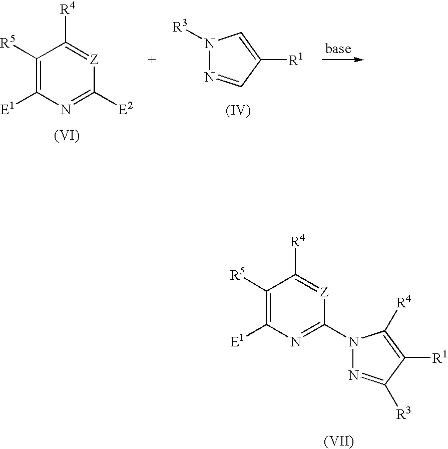 and
d) base-catalyzed reaction of compounds of the formula (VII) with compounds of the formula (VIII), 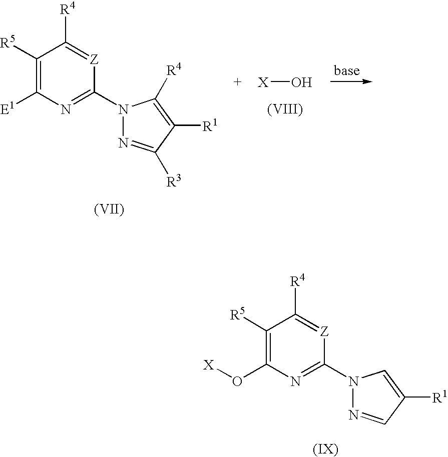 in which
R¹ to R⁵ are various radicals such as alkyl, X are substituted aromatic radicals such as phenyl, and Z is CH or N.

The invention relates to a process for preparing haloalkyl(thio)vinimidinium salts and 4-(haloalkyl(thio))pyrazoles, and to their further conversion to crop protection compositions, especially herbicides and insecticides.

Herbicides and insecticides based on 4-(CF₃)-pyrazole are described in WO-A 99/28301, WO-A 2004/013129, WO-A 2004/013131 and WO-A 02/068413.

These compounds are prepared by reacting, for example, trifluoropropionic acid with a dimethylformamide (DMF)/POCl₃ complex to give CF₃-vinimidinium salts and their further reaction with hydrazine, as described in Tetrahedron Letters V. 37, N 11, pp.1829-1832. The vinimidinium salts are then isolated and purified to free them of excess POCl₃ and other acidic components by flash chromatography on SiO₂, which is impracticable for industrial use.

It is therefore an object of the invention to provide a novel advantageous synthesis for haloalkyl(thio)vinimidinium salts and 4-(haloalkyl(thio))pyrazoles.

It has now been found that, surprisingly, the reaction of (thio)haloalkylpropionic acids with a DMF/phosgene or DMF/(COCl)₂O complex leads in a simple manner to haloalkyl(thio)vinimidinium salts in very good yields and high purity. These salts can then be reacted without isolation in a second step with hydrazine to give 4-(haloalkyl(thio))pyrazoles.

The invention therefore provides a process for preparing vinimidinium salts of the formula (I)

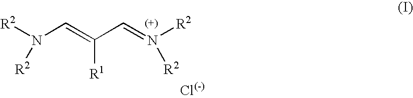

in which

R¹ is (C₁-C₄)-haloalkyl or (C₁-C₄)-haloalkylthio and

R² is (C₁-C₈)-alkyl, (C₃-C₆)-alkenyl, (C₆-C₁₀)-aryl or (C₃-C₈)-cycloalkyl, by reacting a haloalkyl(thio)acetic acid of the formula (II),
R¹—CH₂—COOH (II)

where R¹ is as defined under formula (I)

with a formamide of the formula (III)
H—CO—N(R²)₂ (III)

where R² is as defined under formula (I)

in the presence of ClCOClCl₃C—O—COCl, Cl₃C—O—CO—O—CCl₃ or (COCl)₂ or SOCl₂to give the vinimidinium salt of the formula (I).

The invention further provides a process for preparing a 4-(haloalkyl(thio))pyrazole of the formula (IV)

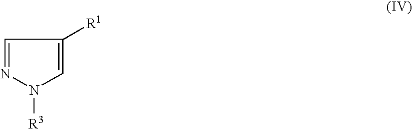

in which

R³ is H, (C₁-C₈)-alkyl, (C₃-C₆)-alkenyl, (C₃-C₈)-cycloalkyl or (C₆-C₁₀)-aryl, by
a) preparing a vinimidinium salt of the formula (I) as specified above and
b) subjecting the vinimidinium salt of the formula (I) thus obtained to a ring-closure reaction with a hydrazine of the formula (V),
   R³NH—NH₂ (V)
in which
R³ is H, (C₁-C₄)-alkyl, (C₃-C₆)-alkenyl, (C₃-C₈)-cycloalkyl or (C₆-C₁₀)-aryl.

In further steps according to Scheme 1 and 2, the compounds of the formula (IV) can be reacted under base catalysis with compounds of the formula (VI) to give compounds of the formula (VII), and these—likewise under base catalysis—can be reacted with compounds of the formula (VIII) to give the compounds of the formula (IX) known as herbicides, for example, from WO 2004/013129 and WO 2004/013131.

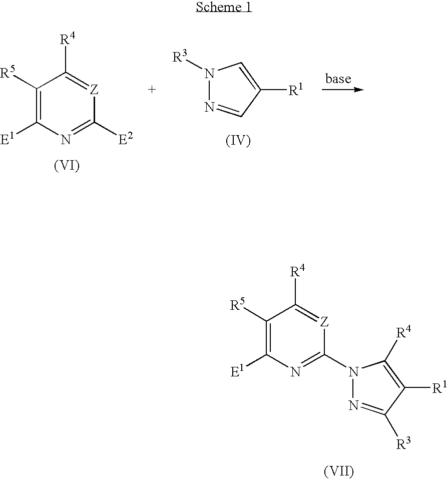

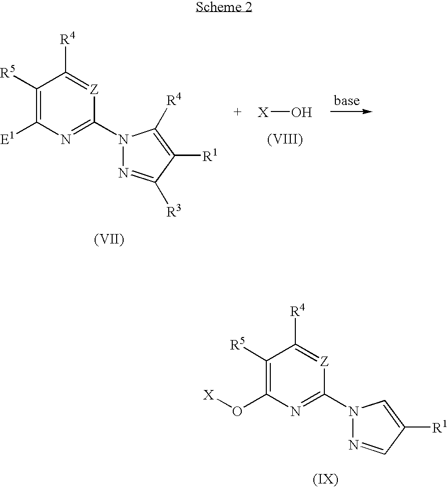

in which
R⁴ and R⁵ are each independently hydrogen, halogen, cyano, OH, (C₁-C₄)-alkyl, halo-(C₁-C₄)-alkyl, (C₃-C₆)-cycloalkyl or (C₁-C₄)-alkoxy.
X is a mono- or poly-trifluoromethyl-, -halo, -methyl-, -cyano, -methoxy- or -trifluoromethoxy-substituted radical from the group of thienyl, pyrazolyl, pyridinyl, phenyl and thiazolyl,
Z is CH or N,

E¹ and E² are each independently a leaving group, such as halogen, methylsulfonyl or tosyl,

R¹ and R³ are each as defined above.

The processes according to the invention enable the preparation of vinimidinium salts of the formula (I), of the 4-(haloalkyl(thio))pyrazoles of the formula (IV) and of the pyrazolylpyridines or pyrazolylpyrimidines of formula (IX) in a simple manner under economically and ecologically advantageous conditions.

The starting compounds for preparing the vinimidinium salts (I), haloalkyl(thio)acetic acids, are known, and their preparation is described, for example, in Tetrahedron Letters V. 37, N 11, p. 1829-32, 1996, WO-02/48111 and Zh. Org. Khim (Russ) 1977, 13(5), 1057-61.

Formamides of the formula (III), phosgene and derivatives thereof, and oxalyl chloride are commercially available or can be prepared by known methods familiar to the person skilled in the art, in the case of the formamides by reacting amines with formic acid derivatives.

The molar ratio of acid (II) to dialkylformamide (III) and phosgene is generally 1:3-10:3-6, preferably about 1:3-3. In the case of phosgene derivatives, such as di- and triphosgene, correspondingly less phosgene derivative is used. In this case, preference is given to using 5 equivalents thereof.

The dialkylformamide preferably also serves as a diluent. When a further diluent is used, it is possible, for example, to use dichloromethane, dichloroethane, Freon113, such as CF₂Cl—CFCl₂, chlorobenzene and/or carbon tetrachloride. It is equally possible to use mixtures of diluents, preferably of the diluents mentioned.

In general, 1-20 weight equivalents of solvent are used per equivalent of acid.

The reaction temperature is generally between 0 and 100° C., preferably between 20 and 75° C.

The reaction time is generally from 1 to 3 hours.

To isolate the vinimidinium salts (I), the excess of diluent(s) is generally removed under reduced pressure. This affords the vinimidinium salts (I) in pure form, and they can optionally be reacted further directly. If desired, the salts (I) can be washed with suitable diluents, such as ether or heptane.

In the reaction of the vinimidinium salts (I) with hydrazines (V) to give the pyrazoles (IV), the molar ratio of vinimidinium salt (I) to hydrazine is generally 1:2-10, preferably 1:3-8, more preferably 1:34.

In one variant of the process, the molar ratio of vinimidinium salt (I) to hydrazine (V) is about 1:1, and 24 equivalents of a further base are added.

Preferred further bases are tertiary amines (especially with (C₁-C₄)-alkyl radicals), hydroxides, (C₁-C₄)-alkoxides of the alkali metals and alkaline earth metals, carbonates and acetates.

Suitable diluents are, for example, i-butyronitrile, toluene, chlorobenzene, acetonitrile and alcohols, especially acetonitrile and ethanol.

The reaction temperature is generally from 20 to 80° C.

The reaction time is generally from 1 to 5 hours.

The pyrazoles (IV) prepared in accordance with the invention can be worked up by known methods familiar to those skilled in the art. For example, the diluent is drawn off under reduced pressure, and the product is extracted, for example with ethyl acetate, washed and, after removal of the solvent, optionally distilled.

The terms used in the formulae (I) to (IV) are explained in detail below:

The term “halogen” means fluorine, chlorine, preferably fluorine and chlorine, more preferably fluorine.

“(C₁-C₄)-Alkyl” is an unbranched or branched hydrocarbon radical having 1, 2, 3 or 4 carbon atoms, for example the methyl, ethyl, propyl, isopropyl, 1-butyl, 2-butyl, isobutyl or tert-butyl radical.

Correspondingly, an alkyl radical with a larger range of carbon atoms is understood to mean an unbranched or branched saturated hydrocarbon radical which contains a number of carbon atoms that corresponds to this range. The expression “(C₁-C₈)-alkyl” accordingly encompasses the aforementioned alkyl radicals, and also, for example, the pentyl, 2-methylbutyl, 1,1-dimethylpropyl, hexyl, heptyl, octyl and tert-octyl radical.

“(C₁-C₄)-Haloalkyl” is an alkyl group mentioned for “(C₁-C₄)-alkyl” in which one or more hydrogen atoms have been replaced by the same number of identical or different halogen atoms, preferably chlorine or fluorine, for example the mono-, di- or trifluoromethyl group, the 1- or 2-fluoroethyl, the 2,2,2-trifluoroethyl, the chloromethyl, trichloromethyl or 1,1,2,2-tetrafluoroethyl group.

“Alkenyl” and “alkynyl” with a preceding range of carbon atoms mean a straight-chain or branched hydrocarbon radical which has a number of carbon atoms corresponding to this range and contains at least one multiple bond, which may be present at any position in the unsaturated radical in question.

“(C₃-C₆)-Alkenyl” is accordingly, for example, the allyl, 2-methylpropenyl, 2- or 3-butenyl, pentenyl, 2-methylenepentenyl or hexenyl group.

“(C₃-C₁₀)-Cycloalkyl” represents monocyclic alkyl radicals such as the cyclopropyl, cyclobutyl, cyclopentyl, cyclohexyl, cycloheptyl or cyclooctyl radical.

“(C₆-C₁₀)-Aryl” represents aromatic groups such as phenyl or naphthyl.

In the formula (I), the R¹ and R² groups are preferably each defined as follows:

R¹ is preferably CF₃.

R² is preferably CH₃.

Preference is given to the compound of the formula (IV) in which

R¹ is CF₃ and

R² is H.

The compounds of the formula (I) and (IV) are valuable intermediates for the synthesis of crop protection compositions, preferably of insecticides and herbicides, especially of compounds which are described in WO-A 99/28301, PCT/EP 0307573 and PCT/EP 0307574.

The invention is illustrated in detail by the examples without thereby restricting it.

### EXAMPLE 1

a) Vinimidinium Salt

Dimethylformamide (59 g) was cooled to 0° C. and phosgene (13 g, 0.135 mol) was introduced slowly. Trifluoropropionic acid (5.8 g, 0.045 mol) was added and the mixture was heated at 70° C. for 1 h. The volatile components were then removed under reduced pressure at 0.1 mbar and 40° C. The ¹⁹F NMR of the mixture showed a purity of the vinimidinium salt formed of 95% (−48 ppm). The salt crystallized when left to stand.

b) 4-Trifluoromethylpyrazole

The salt was dissolved in 120 ml of acetonitrile and hydrazine hydrate (11.7 g) was added dropwise at 20° C. The mixture was then heated to 70° C. for 1 h. The product was isolated by diluting the mixture with water and extracting with ethyl acetate. Yield: 5 g (86%) with a purity of 95%. For a further purification, the product can be distilled under reduced pressure.

4-Trifluoromethlypyrazole ¹H NMR 7.9 s, ppm ¹⁹F NMR −57.2 (CF₃) ppm

1. A process for preparing vinimidinium salts of the formula (I) 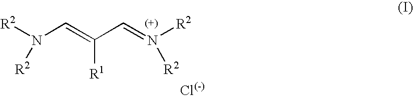
in which
R¹ is (C₁-C₄)-haloalkyl or (C₁-C₄)-haloalkylthio and
R² is (C₁-C₈)-alkyl, (C₃-C₆)-alkenyl, (C₆-C₁₀)-aryl or (C₃-C₈)-cycloalkyl,
by reacting a haloalkyl(thio)acetic acid of the formula (II),
R¹—CH₂—COOH (II)
where R¹ is as defined under formula (I)
with a formamide of the formula (III)
H—CO—N(R²)₂ (III)
where R² is as defined under formula (I)
in the presence of ClCOClCl₃C—O—COCl, Cl₃C—O—CO—O—CCl₃, or (COCl)₂ or SOCl₂ to give the vinimidinium salt of the formula (I).

2. The process as claimed in claim 1, wherein R¹ in the formula (I) is CF₃.

3. The process as claimed in claim 1, wherein the molar ratio of the acid (II) to dialkylformamide (III) is 1:5-3.5.

4. A process for preparing a 4-(haloalkyl(thio))pyrazole of the formula (IV) 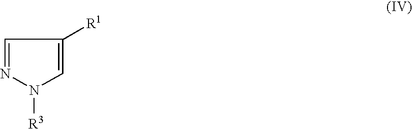
in which
R¹ is (C₁-C₄)-haloalkyl or (C₁-C₄)-haloalkylthio and
R³ is H, (C₁-C₈)-alkyl, (C₃-C₆)-alkenyl, (C₆-C₁₀)-aryl or (C₃-C₈)-cycloalkyl,
by
a) reacting a haloalkyl(thio)acetic acid of the formula (II),
R¹—CH₂—COOH (II)
where R¹ is as defined under formula (I)
with a formamide of the formula (III)
H—CO—N(R²)₂ (III)
where R² is as defined under formula (I)
in the presence of ClCOClCl₃C—O—COCl, Cl₃C—O—CO—O—CCl₃, or (COCl)₂ or SOCl₂ to give the vinimidinium salt of the formula (I),
b) subjecting the vinimidinium salt of the formula (I) thus obtained to a ring-closure reaction with a hydrazine of the formula (V),
R³NH—NH₂ (V)
in which
R³ is H, (C₁-C₄)-alkyl, (C₃-C₆)-alkenyl, (C₃-C₈)-cycloalkyl or (C₆-C₁₀)-aryl.

5. The process as claimed in claim 1, wherein the molar ratio of vinimidinium salt (IV) to hydrazine (V) is 1:2-10.

6. The process as claimed in claim 1, wherein the molar ratio of vinimidinium salt (IV) to hydrazine is about 1:1, and from 2 to 4 equivalents of a base are additionally added.

7. A process for preparing compounds of the formula (IX) 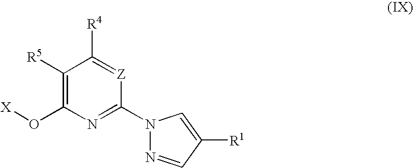
in which
R⁴ and R⁵ are each independently hydrogen, halogen, cyano, OH, (C₁-C₄)-alkyl, halo-(C₁-C₄)-alkyl, (C₃-C₆)-cycloalkyl or (C₁-C₄)-alkoxy.
X is a mono- or poly-trifluoromethyl-, -halo, -methyl-, -cyano, -methoxy- or -trifluoromethoxy-substituted radical from the group of thienyl, pyrazolyl, pyridinyl, phenyl and thiazolyl,
Z is CH or N,
by
a) in a first stage according to claim 1, preparing vinimidinium salts of the formula (I) from haloalkyl(thio)acetic acids of the formula (II) and formamides of the formula (III) 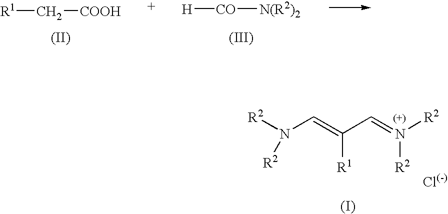
b) in a second stage according to claim 4, preparing 4-(haloalkyl(thio))pyrazoles of the formula (IV) by reacting the vinimidinium salt of the formula (I) with hydrazines of the formula (V) 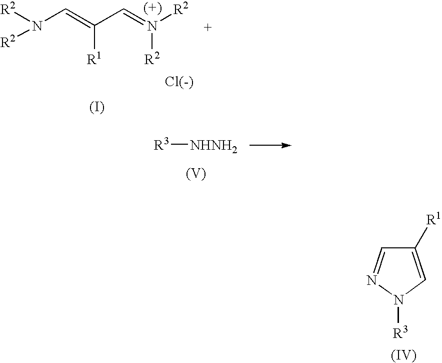
c) in a third stage, reacting compounds of the formula (VI) with pyrazoles (IV) under base catalysis to give compounds of the formula (VII) 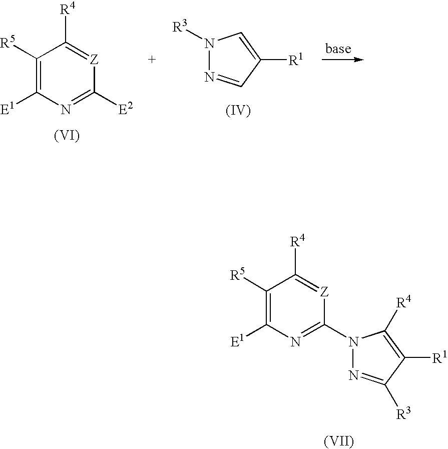
and
d) in a fourth stage, reacting compounds of the formula (VII) with compounds of the formula (VIII) under base catalysis to give compounds of the formula (IX)

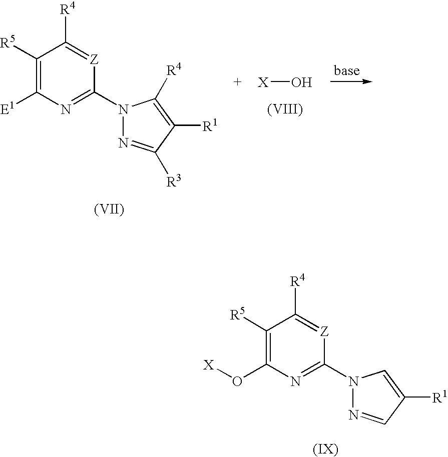

8. A method of use of vinimidinium salts of the formula (I) for preparing pyrazoles of the formula (IV) by reacting them with hydrazines of the formula (V) 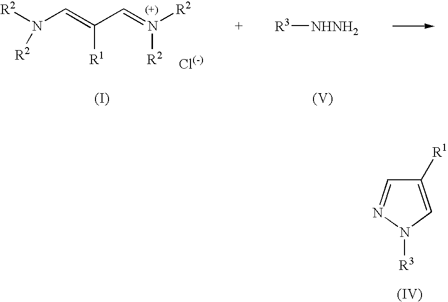
in which R¹, R² and R³ are each as defined in claims 1, 2 and 4.
